(19) United States
(12) Patent Application Publication
(10) Pub. No.: US 2018/0243213 A1
(43) Pub. Date: Aug. 30, 2018

(54) AEROSOLIZED DAPSONE AS A THERAPY FOR INFLAMMATION OF THE AIRWAY AND ABNORMAL MUCOCILIARY TRANSPORT

(71) Applicants: Bruce K. RUBIN, Richmond, VA (US); Soichiro KANOH, Tokyo (JP); Tsuyoshi TANABE, Richmnd, VA (US)

(72) Inventors: Bruce K. RUBIN, Richmond, VA (US); Soichiro KANOH, Tokyo (JP); Tsuyoshi TANABE, Richmnd, VA (US)

(73) Assignee: VIRGINIA COMMONWEALTH UNIVERSITY, RICHMOND, VA (US)

(21) Appl. No.: 15/899,539

(22) Filed: Feb. 20, 2018

Related U.S. Application Data
(63) Continuation of application No. 14/520,976, filed on Oct. 22, 2014, now abandoned.
Application No. 14/520,976 is a continuation of application No. 13/583,434, filed on Sep. 7, 2012, now abandoned.
(60) Provisional application No. 61/416,363, filed on Nov. 23, 2010.
(60) Provisional application No. 61/313,877, filed on Mar. 15, 2010.

(51) Int. Cl. A61K 9/12; A61M 15/00; A61K 9/00; A61K 31/145; A61K 31/136

(52) U.S. Cl. CPC A61K 9/12; A61M 15/009; A61K 9/0073; A61K 31/136; A61K 31/145; A61M 2202/064

(57) ABSTRACT

Aerosolized dapsone (or alternatively, an aqueous formulation of dapsone) is used to treat airway inflammation, particularly chronic neutrophil-dominated inflammation. Diseases that may be prevented or treated by the methods include chronic obstructive pulmonary diseases (COPDs), asthma, cystic fibrosis, and others.

### BACKGROUND OF THE INVENTION

### Field of the Invention

The invention generally relates to the treatment of airway inflammation and conditions and diseases characterized by airway inflammation. In particular, the invention provides aerosolized dapsone (or alternatively, aqueous formulations of dapsone) which, when administered in vivo, causes a decrease in airway inflammation in mammals.

### Background of the Invention

Diseases associated with inflammation of the airways, particularly chronic inflammatory conditions such as asthma, cystic fibrosis, emphysema, and chronic obstructive pulmonary disorder, are frequently debilitating and complicated and costly to treat. Current treatment options for these diseases, which are generally characterized by neutrophil-dominated inflammation, include the use of steroids to suppress the overactivity of the immune system, and the administration of macrolide antibiotics. However, both of these treatments have drawbacks. Steroids suppress the immune system in general, and their use leads to a high risk of infection (e.g. opportunistic infection) in patients. The use of macrolide antibiotics has contributed to the dangerous surge in the evolution of macrolide-resistant bacteria. Clearly, improved strategies for treating airway inflammation are needed.

Dapsone (diamino-diphenyl sulfone), a synthetic sulfone, is successfully used to treat various diseases such as leprosy, Pneumocystis jiroveci (formerly P. carinii) pneumonia and malaria. Dapsone is also recognized as an anti-inflammatory drug and has been used both systemically and topically to treat skin diseases which are characterized by neutrophil-dominated inflammation, e.g. dermatitis herpetiformis (Zhu et al, 2001).

Berlow et al., (1990) described the treatment of steroid-dependent asthma using orally administered dapsone. Patients exhibiting steroid-dependent asthma cannot be weaned from steroid administration without the recurrence of disease symptoms, and yet are at risk for developing side effects from the use of steroids, especially long term. The results of the study showed that 9 out of 10 patients were able to substantially reduce or stop taking steroids while they were taking dapsone. However, oral (and hence, systemic) administration also resulted in significant anemia in 9 out of 10 patients.

Chougule et al. (2008) investigated the development of spray dried liposomal dry powder inhaler formulations of dapsone, with the mention of possibly treating P. carinii infections with such a formulation. The objective of the research was to evaluate deposition of the spray dried formulations in vitro. The results showed that the investigators were able to develop a spray dried formulation of dapsone that exhibited prolonged release (up to 16 hours) across a cellophane membrane when evaluated using a customized diffusion cell. Aerosol performance was also assessed using a commercial Anderson Cascade Impactor device. According to the investigators, the results appeared “promising”. However, these results were highly preliminary; no in vivo testing was attempted, and no effect on inflammation was demonstrated or suggested.

The prior art has thus far failed to provide a method of treating neutrophil dominated airway inflammation using an aerosolized dapsone formulation.

### SUMMARY OF THE INVENTION

The present invention provides a method of treating inflammation of the airways, particularly neutrophil-dominated inflammation, using aerosolized (or alternatively, an aqueous) formulations of dapsone. The present invention is the first to demonstrate that dapsone, when administered to a mammal in this manner, causes resolution (e.g. a decrease, lessening or lowering) of the symptoms associated with neutrophil-dominated inflammation in the airways of an afflicted individual. The present invention also includes the first demonstration of the mode of action of dapsone: dapsone functions as an immune modulator, rather than as an immune suppressor. Thus, the administration of dapsone in lieu of e.g. steroids to treat inflammation is less likely to increase the risk of infection in a patient receiving the treatment. Further, since the compound does not exert selective pressure on microbes, the use of dapsone does not contribute to the rise in antibiotic resistant bacterial strains. Significantly, as demonstrated herein, both oral and aerosol dapsone decreased LPS-induced intraepithelial neutrophil accumulation, but only treatment with aerosol dapsone restored mucociliary transport to normal (and at lower concentrations that are required for oral administration). Further, this targeted approach to the delivery of dapsone is less likely to cause the untoward side effects that result from oral, systemic dapsone delivery (e.g. anemia).

### BRIEF DESCRIPTION OF THE DRAWINGS

FIG. 1A-C. Effect of dapsone on IL-8 secretion from NHBE cells in culture. Growth factors were withdrawn from the culture medium 24 h before LPS or dapsone exposure, and supernatants were harvested 24 h after LPS stimulation. A: LPS 10 μg/ml significantly increased IL-8, and dapsone 0.3, 1 or 10 μg/ml suppressed this effect. B: Dapsone 1 μg/ml did not influence basal IL-8 secretion at 24, 48 and 72 h. C: Dapsone 1 μg/ml inhibited LPS-induced IL-8 secretion to the control level at 24 and 72 h. Values are means±SE. n=6. *P<0.05, ***P<0.001 compared with control (Cont). #P<0.05, ##P<0.01 compared with LPS alone.

FIG. 2A-D. Effect of dapsone and dexamethasone (DEX) on LPS-induced apical (A, C) or basolateral (B, D) IL-8 secretion from NHBE cells cultured under air-liquid interface condition. NHBE cells were incubated in medium with and without dapsone 1 μg/ml (A, B) or DEX 0.1 μg/ml (C, D), and stimulated with LPS 10 μg/ml for 24 h from the apical (AP) or basolateral (BL) side. A: AP-LPS significantly increased apical IL-8 secretion, an effect that was inhibited by dapsone. B: AP- and BL-LPS significantly increased basolateral IL-8 secretion, an effect that was inhibited by dapsone. Values are means±SE, n=6 for dapsone. ***P<0.001 compared with control. #P<0.05, ###P<0.001 compared with LPS alone. C: AP-LPS significantly increased apical IL-8 secretion. DEX inhibited LPS-induced IL-8 secretion as well as the basal IL-8 level. D: AP- and BL-LPS significantly increased basolateral IL-8 secretion, an effect that was inhibited by DEX. DEX also inhibited basal IL-8 level. Values are means±SE, n=4 for DEX. *P<0.05, **P<0.001 compared with control. #P<0.05 compared with LPS alone.

FIG. 3. Effect of dapsone on LPS-induced IL-8 mRNA expression. Growth factors were withdrawn from the culture medium 24 h before LPS, dapsone or dexamethasone (DEX) exposure. NHBE cells were stimulated with LPS 10 μg/ml, dapsone 0.3-10 μg/ml, DEX 0.1 μg/ml or their combination for 4 h, and IL-8 mRNA expression was evaluated using real-time quantitative PCR. Dapsone 1 μg/ml did not influence basal IL-8 mRNA level, but DEX reduced this. LPS 10 μg/ml increased IL-8 mRNA expression more than 5-fold, an effect that was inhibited by dapsone 1 and 10 μg/ml and by DEX 0.1 μg/ml. Data are expressed as fold change compared to control. Values are means±SE. n=4. *P<0.05, ***P<0.001 compared with control. #P<0.05, ##P<0.01 compared with LPS alone.

FIG. 4. Temporal effect of dapsone on LPS-induced MAPK activation over 24 h. Growth factors were withdrawn from the culture medium 24 h before LPS or dapsone exposure. Threonine and tyrosine phosphorylation of ERK1/2, p38 and JNK was measured by Western blotting. The band intensity was calculated with NIH Image J software. LPS 10 μg/ml increased the ratio of phospho (p)-ERK1/2/ERK1/2, but not p-p38/p38. p-JNK was not detected. Dapsone 1 μg/ml inhibited LPS-induced ERK1/2 phosphorylation at 1 h, but not at 4 and 24 h. Values are means t SE from more than three independent experiments. *P<0.05 compared with control (LPS−, dapsone−, at each time). #P<0.05 compared with LPS alone.

FIGS. 5A and B. Effect of PD98059 (MEK inhibitor) on LPS-induced ERK1/2 phosphorylation (A) and IL-8 secretion (B). Growth factors were withdrawn from the culture medium 24 h before LPS exposure. PD98059, 20 μM was added 1 h before LPS stimulation. A: LPS increased ERK1/2 phosphorylation in a dose-dependent manner at 4 h, an effect that was abolished by PD98059. Values are means±SE from four independent experiments. *P<0.05, **P<0.01 compared with control (Cont). #P<0.05 compared with LPS alone. B: PD98059 did not inhibit LPS-induced IL-8 secretion. Values are means+SE, n=6. ***P<0.001 compared with control.

FIGS. 6A and B. Effect of dapsone treatment on LPS-induced neutrophil accumulation in ferret airways. Ferrets were intubated with an LPS (10 μg)-coated endotracheal tube for 30 min once daily for 5 days, and dapsone was administered orally or in nebulized form from day 4 to day 8. Tracheas were removed on day 9, and histological analyses were performed. The total number of intraepithelial neutrophil was counted over 150 μm in eight random sites per specimen from four different sections and averaged. A: Oral dapsone decreased intraepithelial neutrophil number, but not significantly. Values are means±SD. n=4. B: Nebulized dapsone significantly inhibited neutrophil accumulation. Values are means+SD. n=4 for vehicle and n=5 for dapsone. *P<0.05 compared with vehicle (LPS+, dapsone−).

FIGS. 7A and B. Effect of dapsone treatment on LPS-induced inhibition of mucociliary transport (MCT) timed over a 3 mm segment. A: Oral dapsone increased MCT, but not significantly (P=0.09). Values are means+SD. n=4. B: Nebulized dapsone significantly increased MCT (P=0.007) to normal levels. Values are means+SD. n=5. **P<0.01 compared with vehicle (LPS+, dapsone−).

### DETAILED DESCRIPTION

The invention provides methods of treating inflammation of the airways by administering aerosol formulations of dapsone. Without being bound by theory, it is believed that dapsone exerts an immunomodulatory (as opposed to an immunosuppressive) effect by inhibiting IL-8 and IL-13. IL-8, a member of the cysteine-X-cysteine (CXC) chemokine family, acts as one of the most potent neutrophil chemoattractants. Hence, attenuation of IL-8 activity lessens or decreases the recruitment of neutrophils to a site of inflammation, thereby decreasing or lowering neutrophil-dominated inflammation at the site. IL-13 is known to induce goblet cell hyperplasia in asthmatics, and inhibition of this process also aids in controlling the symptoms of airway inflammation.

The methods of the invention are advantageous compared to the use of steroids to counter inflammation, because steroids are immunosuppressants and, while their use may decrease inflammation, their use also results in immunosupression, thereby increasing the risk of infection (e.g. opportunistic infection). Likewise, the use of dapsone is advantageous compared to the use of macrolide antibiotics, since the use of dapsone does not contribute to the evolution of macrolide-resistant bacteria. In addition, as demonstrated herein, while both oral and aerosol dapsone decreased LPS-induced intraepithelial neutrophil accumulation, but only treatment with aerosol dapsone restored mucociliary transport to normal. (Mucociliary clearance, the self-clearing mechanism of the bronchi that is carried out by cilia which are present on the respiratory epithelium, is an indicator of the health of the airway surface.) Thus, the finding that aerosolized dapsone is superior to orally administered dapsone in restoring this important function is of great consequence.

In an alternative embodiment, the dapsone is administered to the airways via, for example, installation of an aqueous, physiologically acceptable carrier comprising dapsone, described below.

Practice of the method of the invention results in a decrease in symptoms of inflammation in the airways of a subject treated with an aerosolized dapsone preparation. Administration results in inhibition of IL-8 and restoration of mucociliary transport and clearance. The decrease (lessening, amelioration, resolution, etc.) may be complete (i.e. symptoms may entirely disappear) but this need not always be the case. Those of skill in the art will recognize that much benefit can be accrued for a patient in whom symptoms are only lessened or partially ameliorated, e.g. to a level which allows the patient to resume a normal or near-normal level of activity. Those of skill in the art are familiar with the assessment and measurement of the effect of such treatments, e.g. by measuring lung capacity, extent of airway occlusion, blood oxygenation levels, by observing the presence/absence and/or frequency of symptoms (e.g. wheezing, coughing, etc.), various imaging techniques, and others. Generally, the practice of the methods of the invention leads to at least about a 10, 20, 30, 40, 50, 60, 70, 80, 90 or even 100% reduction in symptoms of inflammation.

The methods of the invention involve administering physiologically compatible aerosol compositions of dapsone (or, in an alternative embodiment, dapsone in an aqueous carrier) to the respiratory system of a patient or subject. The phrase “respiratory system” is intended to include all orifices and passages that participate in carrying air (usually oxygen-rich air) to and from the lungs and waste, CO₂ rich air from the lungs, as well as the lungs themselves. For example, included are the nose and nasal cavities, the mouth, the larynx, trachea, bronchi and bronchiole tubes and their branches, and alveoli small airways (e.g. membranaceous bronchioles, which are noncartilaginous conducting airways with a fibromuscular wall; and respiratory bronchioles, which are airways in which the fibromuscular wall is partially alveolated). Two natural orifices through which aerosolized dapsone can be administered are the nose and mouth, and administration via either or both of these is encompassed by the invention. However, the aerosols may also be delivered through surgically introduced openings (e.g. tracheotomies), or even directly to, for example, the lungs e.g. via intubation.

As such, the delivery may be accomplished by using any of many known aerosol administering devices, including but not limited to mouth inhalers (dry powder inhalers, metered dose inhalers, etc.), face masks, intranasal or intra-tracheal tubes, nebulizers, etc. The type of device that is selected will vary according to the circumstances, e.g. whether the aerosol is self-administered by the patient, or whether in e.g. situations of acute attacks or crises, delivery is carried out by medical personnel. Generally, for the treatment of chronic disease, the devices that are used will be suitable for patient self-administration. Such administration may be carried out using any of several types or styles of aerosol delivery devices known in the art. Exemplary devices include but are not limited to metered-dose inhalers (MDIs, e.g. “puffers”), in which medication is most commonly stored in solution in a pressurized canister that contains a propellant (e.g. fluorocarbons such as 134a or 227, pressurized air, alkanes, etc.), although it may also be a suspension; dry powder inhalers, (DPIs) which release a dose of medicine as a powder aerosol; and nebulizers, which supply the medication as an aerosol created from an aqueous formulation. The devices may be, for example, single-dose or multi-dose, disposable or reusable/refillable, etc., and may be made from a variety of materials and in a variety of shapes, and may operate by a variety of mechanisms (e.g. breath-hold, breath-actuated, etc.).

In some embodiments, inhalation devices for use in the present invention are breath actuated, i.e. delivery of the aerosolized formulation is restricted o the period of actual inhalation by the patient. One such breath-actuated representative inhalation device suitable for use in the practice of the invention is the Aerodose™ inhaler, available from Aerogen, Inc., Sunnyvale, Calif. This inhaler generates an aerosol using a porous membrane driven by a piezoelectric oscillator. Other inhaler or nebulizer devices that may be employed include conventional air-jet nebulizers, for example, the PARI LC PLUS™ jet nebulizer (PARI GmbH, Stamberg, Germany); and others that are known in the art. Various systems, devices and compositions for the delivery of aerosols are described, for example, in the following issued US patents, the complete contents of each of which is herein incorporated by reference: U.S. Pat. Nos. 7,740,463; 7,683,029; 7,497,214; 7,223,381; 7,163,014; 7,040,314; 6,932,962; 6,743,413, and 6,575,162.

As used herein, the term “aerosol” refers to a suspension of solid or liquid particles in a gaseous medium. Herein, this term also encompasses, for example, “mists”, “nebulized formulations”, etc. The formulations that are administered according to the present invention are suitable for aerosolized delivery to a patient in need thereof. Thus, the formulations are physiologically compatible. At a minimum, the formulations contain dapsone plus a physiologically/biologically compatible or suitable carrier. The amount of dapsone in a formulation may vary, but is generally in the range of from about 1 to 99% (wt/vol).

Formulations suitable for delivery to the lung of a patient generally comprise either solid particles which comprise dapsone, suspended in a gaseous medium when delivered, or liquid droplets comprising dapsone, suspended in a gaseous medium when delivered. Commercial sources of dapsone are well-known to those of ordinary skill in the art. Those of ordinary skill in the art are also well acquainted with the production and manufacture of such formulations, which may, in addition to the active agent dapsone, include one or more additional components. If a liquid carrier is used, it may be sterile saline or saline buffered at a physiologically compatible pH (e.g. from about 6.5 to 8.0, usually about 7.3-7.4). Exemplary additional components include but are not limited to: stabilizers, preservatives, various organic and inorganic pharmaceutical excipients, including various polymers, low molecular weight oligomers, natural products, wetting agents, and surfactants, in particular, nonionic and ionic surfactants. Representative examples of additional components (which may be surface modifiers) include cetyl pyridinium chloride, gelatin, casein, lecithin (phosphatides), dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens such as e.g., polysorbate 20, commercial name Tween® 20 and Tween 80® (ICI Specialty Chemicals)); polyethylene glycols (e.g., Carbowaxs 3350@ and 1450®, and Carbopol 934® (Union Carbide)), dodecyl trimethyl ammonium bromide, polyoxyethylene stearates, colloidal silicon dioxide, phosphates, sodium dodecylsulfate, carboxymethylcellulose calcium, hydroxypropyl cellulose (HPC, HPC-SL, and HPC-L), hydroxypropyl methylcellulose (HPMC), carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); a charged phospholipid such as dimyristoyl phophatidyl glycerol, dioctylsulfosuccinate (DOSS); Tetronic 1508® (T-1508) (BASF Wyandotte Corporation), dialkylesters of sodium sulfosuccinic acid (e.g., Aerosol OT®, which is a dioctyl ester of sodium sulfosuccinic acid (American Cyanamid)); Duponol P®, which is a sodium lauryl sulfate (DuPont); Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-1OG® or Surfactant 10-G® (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; and the like. Tyloxapol is a particularly preferred surface modifier for pulmonary or intranasal delivery, even more so for nebulization therapies. Most of these compounds are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), specifically incorporated by reference. They are commercially available and/or can be prepared by techniques known in the art.

In addition, the dapsone aerosol may contain and be formulated with other biologically active components, e.g. other compounds with anti-inflammatory (or other) properties such as steroids, antibiotics (e.g. macrolides), decongestants, anti-cancer agents, etc. Alternatively, the aerosol dapsone formulation may be used in conjunction with such biologically active components, although they are not included in the same formulation.

The delivery schedule that is maintained by the patient (or a health care professional that is caring for the patient) may vary depending on several factors, e.g. the disease or condition that is being treated, the severity of the condition; the age, gender and weight of the patient; convenience of scheduling in order to achieve or maximize compliance, etc. Generally, administration takes place from 2-4 times daily (e.g. about every 12 hours, or every 8 hours, or every 4 hours), but may also be less frequent (e.g. only once per day) or more frequent (e.g. every 2 hours) in some cases. The duration of each administration may differ, depending on the amount that is to be delivered, the type of device that is used, etc. Generally, administration requires from about 5-30 minutes, e.g. about 5, 10, 15, 20, 25 or 30 minutes. However, some rapid delivery devices may accomplish delivery in only a few (e.g. 1-4) minutes. Further, for some patients (e.g. a patients who is experiencing an acute asthma attack) administration may be continuous and ongoing for a longer period of time, e.g. until the patient is transported to a hospital setting, or until the patient is stabilized in a medical setting, etc. A skilled medical professional (e.g. a doctor, respiratory therapist, etc.) is well aware of these factors and will be able to plan and adjust treatment protocols accordingly.

The amount of dapsone that is delivered per dose will also vary according to various factors, e.g. the disease or condition that is being treated, the severity of the condition; the age, gender and weight of the patient; tolerance of the patient for the treatment; etc. Generally, the amount administered by inhalation will range from about 0.5 to about 5 mg/kg of body weight, e.g. from about 0.5 to about 1.0, 1.5, 2.0, 2.5, 3.0, 3.5, 4.0, 4.5 or 5.0 mg/kg of body weight, and frequently will be about 2 mg/kg of body weight, in a single dosing session.

Diseases which can be treated using the methods of the invention include various inflammations of the airways, especially neutrophil dominated inflammations, which include but are not limited to various obstructive lung diseases in which the bronchial tubes become narrowed making it hard to move air in and especially out of the lung, for example: Chronic Obstructive Pulmonary Disease (COPD) and asthma (ongoing chronic asthma, severe asthma, and asthma “flare-ups” or acute attacks, especially neutrophilic severe asthma); cystic fibrosis; bronchiectasis, bronchiolitis obliterans; pulmonary fibrosis (e.g. idiopathic pulmonary fibrosis); emphysema; acute respiratory distress syndrome; bronchitis; chronic bronchitis; chronic sinusitis; rhinosinusitis and chronic rhinosinusitis; chronic airway infection; toxic inhalation injury; etc. The inflammation can be caused by any of many triggers, including but not limited to tobacco smoking or exposure to second hand smoke; occupational exposure to workplace dusts found in coal mining, gold mining, and the cotton textile industry and chemicals such as cadmium, isocyanates, and fumes from welding; exposure to air pollution (e.g. sulfur dioxide, carbon monoxide, particulates such as soot, dust, etc.); indoor air pollution e.g. from cooking fire smoke and fireplace smoke; genetic susceptibility (e.g. alpha 1-antitrypsin deficiency, and autoimmune reactions (e.g. sustained inflammation mediated by autoantibodies and autoreactive T cells); allergic immune reactions and/or anaphylaxis caused by e.g. dust mites, pet dander, pollen, foods, insect bites or stings, etc.; and others.

The methods of the invention involve administering an aerosolized formulation of dapsone to a patient suffering from inflammation of the airways. Such patients are generally mammals, usually humans, but this need not always be the case. Veterinary applications of the technology are also contemplated.

The methods of the invention generally involve identification of a patient that is suffering from a disease or condition characterized or caused by airway inflammation or abnormal mucociliary transport (MCT) (e.g. slower than normal or basal level MCT), especially inflammation in which neutrophils play a role. Exemplary diseases include but are not limited to cystic fibrosis, bronchiectasis, bronchiolitis obliterans, emphysema, chronic bronchitis, chronic rhinosinusitis, toxic inhalation injury, chronic obstructive pulmonary disease, idiopathic pulmonary fibrosis, asthma, and chronic airway inflammation. The methods are implemented by administering dapsone to the airways of an affected patient, e.g. in order to contact airway epithelial cells, especially epithelial cilia. The methods of the invention generally result in a lowering or decrease in IL-8 overexpression (i.e. the methods prevent expression of IL-8 mRNA at levels which are above normal, control or basal levels). As a result, disease symptoms caused by such overexpression abate in patients suffering from diseases associated with IL-8 overexpression. For example, mucociliary transport returns to normal or near-normal levels. Those of skill in the art are familiar with methods and tests for assessing, identifying and/or diagnosing such patients by observing and/or measuring certain parameters, e.g. breathing characteristics; imaging analyses of lungs and airways; blood levels of oxygen, CO₂, etc; patient self-reporting; biopsy; sputum analysis; and others. Once a patient who is a likely candidate for aerosol dapsone therapy has been identified, a medical professional will generally prescribe a dose and/or dosing regimen for the patient, as well as providing instructions and possibly teaching regarding or demonstrations of the use of an inhaler. The medial professional will then monitor the outcome of administration of the aerosol. Doses and/or dosing frequency may be adjusted according to the patient's reaction or response to the therapy and the progress that is made toward controlling or resolving the clinical symptoms of disease. The duration of therapy may vary from patient to patient, or for an individual patient at different times, and may be short-term or long-term. Frequently, due to the chronic nature of the conditions being treated, aerosol dapsone therapy is long term and continues e.g. for weeks, or months, or years, or even for the remainder of the patient's life.

Those of skill in the art will recognize that delivery of an active agent to the airways of a patient need not always be accomplished using an aerosolized formulation. For example, installation of a drug (e.g. through an existing conduit such as a tracheotomy tube, nasal tube, etc.) may require the use of an aqueous formulation, e.g. dapsone in a physiologically acceptable liquid carrier. Those of skill in the art are knowledgeable in the preparation of such formulations, which share many properties with aerosol preparations as described above for aerosols (e.g. amount of active agent and excipients present in a preparation; pH; diseases treated; identification, diagnosis and monitoring of patients; administration schedules; administration with other agents; etc.). However, such preparations are not “dry powders” but liquids. Routes and methods of administration of such formulations include but are not limited to, for example: installation; via manually dispensed nasal mists or sprays (e.g. delivery by manual squeezing or pumping of a container or device); via nose drops or nasal irrigation; etc.

### EXAMPLES

### Example 1. Dapsone Inhibits IL-8 Secretion from Human Bronchial Epithelial Cells Stimulated with LPS and Resolves Airway Inflammation in the Ferret

### Introduction

The respiratory tract is lined with epithelial cells that separate the internal milieu of the host from the outside world. Airway epithelia are not only a mechanical barrier to external stimuli and microbes but are actively involved in the innate and acquired immune responses and airway inflammation. In response to bacterial invasion, mucociliary clearance is stimulated and inflammatory mediators and cytokines are secreted as a defense but these can also damage the airway. Among epithelial-derived pleiotropic cytokines, IL-8, a member of the cysteine-X-cysteine (CXC) chemokine family, acts as one of the most potent neutrophil chemoattractants. Neutrophil-dominated inflammation is characteristic of chronic obstructive pulmonary disease (COPD), diffuse panbronchiolitis (DPB) and cystic fibrosis (CF). IL-8 is produced by airway epithelial cells. Increased IL-8 in sputum and bronchoalveolar lavage (BAL) fluid is associated with the severity of DPB and CF and there is increased IL-8 gene expression in the bronchial epithelium of subjects with severe asthma and COPD.

Pro-inflammatory cytokines, bacterial flagellin and lipopolysaccharide (LPS) can increase IL-8 production by normal human bronchial epithelial (NHBE) cells. Among the many agents present in organic dusts, LPS, is a major inducer of the inflammatory reaction. LPS binds to toll-like receptor 4 (TLR4), which activates intracellular signaling pathways, including the nuclear factor-cB (NK-κB) pathway, the phosphatidylinositol 3-kinase (PI3K) and, mitogen-activated protein kinase (MAPK) pathways. Three MAPK pathways contribute to IL-8 gene expression, the extracellular-regulated protein kinase (ERK), c-Jun NH2-terminal protein kinase (JNK), and p38 MAPK cascades. The relative degree of activation of each of these pathways and the functional consequences differ among cell types and experimental systems.

Macrolides antibiotics decrease neutrophils and IL-8 concentration in BAL from subjects with DPB, and sputum IL-8 concentration in CF. Macrolides can inhibit IL-8 release from airway epithelial cells in culture through inactivation of ERK or NK-κB.

We hypothesized that dapsone would inhibit IL-8 secretion by stimulated airway cells. We therefore studied the effect of dapsone on IL-8 secretion from NHBE cells stimulated with LPS and further investigated the signaling pathways involved. We then evaluated in the effectiveness of dapsone in decreasing airway neutrophil recruitment and preserving mucociliary clearance when administered orally or as an aerosol to ferrets with airways that had been cxposed to (inflamed by) LPS.

### Material and Methods

### Reagents

Dapsone (4,4′-diaminodiphenyl sulfone), LPS (Escherichia coli serotype 0111: B4), and all other reagents were purchased from Sigma-Aldrich Co. (St. Louis, Mo.) unless otherwise indicated. PD-98059, a MAPK/ERK kinase (MEK, an upstream kinase of ERK1/2) inhibitor was obtained from Calbiochem (La Jolla, Calif.). Phospho- and non-phospho-specific ERK1/2, anti-p38 MAPK, anti-SAPK/JNK, and phospho-specific NF-κB p65 (Ser536) as well as anti-rabbit-IgG HRP antibodies were purchased from Cell Signaling Technology (Beverly, Mass.). DMSO was used as a solvent of dapsone, and the final concentration did not exceed 0.01% (v/v). Preliminary in vitro experiments showed that 0.01% DMSO-medium had no significant effect on cell viability and IL-8 secretion for up to 72 h (data not shown).

### NHBE Cell Culture

NHBE cells (Lonza Walkersville, Walkersville, Md.) were plated at 3,500 cells/cm² in culture dishes in bronchial epithelial cell growth medium (BEGM) supplemented with the SingleQuot® kit (Lonza) without antibiotics and cultured at 37° C. in a 5% CO₂ incubator. We used endotoxin-free media (<0.005 endotoxin units/ml) and second-passage cells for all experiments. Cells were grown to confluence for 6 days. Cultures without antibiotics were then transferred to 6-well or 35 mm dishes coated with type 1 rat-tail collagen and seeded at 3,500 cells/cm². The medium was changed every 24 h. To avoid influence of growth factors on cell signaling and IL-8 secretion, cells were cultured in supplement-free bronchial epithelial cell basal medium (BEBM) for 24 h before stimulation. We evaluated cell response at the time of cell confluence rather than normalize to the relative number of cells because cell maturation could affect cell signaling and cytokine secretion, and at confluence, all cells are at similar growth stages.

For NHBE cell differentiation, cells were plated at 2.0×10⁵ cells/cm² onto polycarbonate inserts of 6.5-mm diameter, 0.4-μm pore size and 10-μm thickness (Costar Transwell Clear, Cambridge, Mass., USA) coated with type 1 rat-tail collagen, and cultured with serum-free DMEM/F12 medium containing ITS-A (1.0%; Invitrogen Co., Carlsbad, Calif.), epidermal growth factor (EGF) (recombinant human EGF, 0.5 ng/ml; Invitrogen Co.), triiodothyronine (10 ng/ml; MP Biomedicals, Solon, Ohio), hydrocortisone (0.5 μg/ml; MP Riomedicals), all-trans retinoic acid (1.0×10-7 M; Sigma-Aldrich), bovine serum albumin (2.0 μg/ml; Sigma-Aldrich) and bovine pituitary extract (30 μg/ml; Invitrogen Co.). After achieving confluence, the apical medium was removed, and cells were cultured with an air-liquid-interface (ALI) method. The culture medium was changed every 48 h, and cells were maintained at 37° C. in a 5% CO₂ for 10-14 days.

### Cytotoxicity Assay

To determine the number of viable cells, formazan dye generation was measured using Cell Counting Kit-8 (CCK-8; Dojindo, Kumamoto, Japan). Cells treated with CCK-8 assay solution were incubated for 2 h and the absorbance at 450 nm was measured with a microplate reader. Data were expressed as % of control cells that were not exposed to dapsone.

### Measurement of IL-8 Secretion

Culture supernatants were collected and centrifuged for 5 min at 200×g and stored at −20° C. until assayed. IL-8 was measured by ELISA (Beckman Coulter, Inc., Brea, Calif.) according to the manufacturer's instructions. Concentrations in each sample were obtained by interpolation from standard curves, and calculated as the mean of the results at the sample dilution.

### Immunoblotting

After stimulation, the plated cells were washed with cold PBS, and then lysed on ice in a modified radio immunoprecipitation buffer (1% Nonidet P-40, 1% sodium deoxycholate, 150 mM NaCl, 10 mM Tris pII 7.5, 5 mM sodium pyrophosphate, 1 mM NaVO4, 5 mM NaF, 1 g/ml aprotinin, 1 μg/ml leupeptin, and 0.1 mM PMSF) for 15 min and then scraped from the dishes. DNA was sheared by passing the lysate though a 27-gauge needle, and insoluble material was removed by centrifugation at 20,000 g for 15 min at 4° C. The protein concentration of the resulting supernatant was quantified by the DC protein assay (Bio-Rad, Hercules, Calif.). Equal amounts of protein extracts were loaded on a 12% SDS-PAGE mini gel and transferred to a nitrocellulose membrane (Bio-Rad). Membranes were blocked with blocking buffer (150 mM NaCl, 20 mM Tris, and 0.1% Tween 20, pH 7.6) containing 5% nonfat dry milk at 4° C. overnight. Subsequently, membranes were rinsed and incubated with the primary antibody: phospho (p)-p44/42 MAPK (Thr202/Tyr204) (diluted 1:2000), p-p38 MAPK (Thr180/Tyr182) (diluted 1:1000), p-SAPK/JNK (Thr183/Tyr185) (diluted 1:1000), or p-NF-κB p65 rabbit polyclonal IgG (diluted 1:1000) (Cell Signaling Technology), for 2 h at room temperature. The membranes were then incubated at room temperature for 1 h with the anti-rabbit IgG HRP secondary antibody (diluted 1:2000). Subsequently, the blots membranes were developed with LumiGLO chemiluminescent substrate peroxide (Cell Signaling Technology).

Membranes were stripped with a stripping buffer (100 mM 2-mercaptoethanol, 2% SDS, and 62.5 mM Tris/HCl pH 6.7) for 30 min at 30° C. The blots were reprobed with anti-p44/42 MAPK, anti-p38 MAPK, or anti-SAPK/JNK antibody (diluted 1:1000 for each), followed by anti-rabbit-TgG HRP secondary antibody (diluted 1:2000). To correct small differences in loading for NF-κB p65, blots were stripped, and reprobed with anti-human β-actin antibody (diluted 1:5000), followed by anti-mouse IgG HRP secondary antibody (diluted 1:5000). Western blot images were scanned and analyzed on NIH Image J software (18).

### Real-Time Quantitative Polymerase Chain Reaction

For the relative quantification of IL-8 mRNA expression, the expression of glyceraldehyde-3-phosphate dehydrogenase (GAPDH) was served as an internal control.

EvaGreen was used as a DNA intercalator dye to monitor amplified DNA quantification, and real-time quantitative PCR curves were analyzed by the CFX Manager™ software (Bio-Rad) in order to obtain threshold cycle (Ct) values for each sample. Quantification was based on a standard curve. Appropriate IL-8 and GADPH forward and reverse primers were used.

### Animal Study

Twenty adult male ferrets (weight, 1.3 to 2.0 kg) were obtained from Marshall Farms (Rose, N.Y.). Ferrets were anesthetized with 40 mg/kg ketamine and 5 mg/kg xylazinc, and exposed to endotoxin for 30 min once daily for 5 days by intubating with a 3.0 uncuffed endotracheal tube (ETT) coated with 10 mg of LPS mixed in 300 μl water soluble K-Y jelly (Johnson & Johnson, New Brunswick, N.J.) (Abanses et al., 2009). As controls, K-Y jelly without LPS was used in two ferrets.

Dapsone was prepared in a vehicle of 5% (v/v) ethanol/5% (v/v) dimethylsulfoxide (DMSO)/0.5% (w/v) methyl-cellulose solution for oral administration in a final concentration of 3 mg/ml. To administer in nebulized form, dapsone was dissolved in 0.67% (v/v) DMSO/saline at a concentration of 0.5 mg/ml. Eighteen LPS-treated ferrets were randomized to receive 5-day dapsone treatment starting on day 4 (after 3 days of LPS) and continuing for 5 days. For oral administration, 2 mg/kg dapsone (n=4) or vehicle alone (n=4) was given daily through a nasogastric tube. This is equivalent to the oral dose used to treat persons with skin disease. For aerosol inhalation, ferrets were shallowly intubated with ETT, which was placed 1 cm below the larynx. Ferrets were treated with nebulized 0.5 mg/ml dapsone (n=5) or 0.67% DMSO/saline (vehicle) (n=5) for 15 min once daily using a jet nebulizer (PariMaster; PARI Respiratory Equipment Inc., Richmond, Va.). Ferrets (body weight 1.5 kg) have 0.3-0.4 ml total lung lining fluid volume. Therefore, the dose delivered to the lung, is estimated to be 0.1 to 1.0 μg in this system given reported nebulization efficiency.

On day 9 dapsone-treated and vehicle control animals were sacrificed and the tracheas removed. Each tracheal segment was fixed in 10% formalin, embedded in paraffin, and processed for histological analysis using a light microscope (CKX41; Olympus, Tokyo, Japan) and by photography using a digital camera system (AxioCam ICc 1; Carl Zeiss, Thomwood, N.Y.). The tissue was cut in 4-μm thickness, and the slides were stained with hematoxylin and eosin. To measure the accumulation of inflammatory cells and overall severity of inflammation, the total number of intraepithelial neutrophil was counted over 150 μm in eight random sites per specimen from four different sections and averaged.

We also used an excised tracheal segment to measure tracheal mucociliary transport (MCT) velocity. Cilia transport mucus loaded e.g. with foreign particles and microorganisms towards the mouth, where it is either swallowed or expelled via coughing. Under conditions of inflammation, the ciliary cells suspend their transport function and bacterial germinal colonization, further irritation and inflammation is facilitated.

A tracheal secgment was placed on a piccc of gauze saturated with Ringer's solution in a chamber in which the relative humidity was maintained at 95 to 100% and the temperature was maintained at 22° C. to 24° C. MCT was measured by focusing a tracheal segment under a microscope with gradicule (grid line) eyepiece and recording the transport time of the leading edge of very fine shavings of plastic that were placed on the tracheal epithelium. The time to transport the particle 3 mm was used to calculate the MCT (in millimeters per minute). Measurements were repeated five times for each tracheal segment. This study was approved by the Animal Care and Use Committee of Virginia Commonwealth University.

### Statistics

Results are expressed as means values±SE or SD as appropriate. Statistical analysis of data was performed with the StatView 5 statistics package (SAS Institute, Cary, N.C.). Comparisons between two groups were made by unpaired Student's t test. Multiple comparisons were made by one-way analysis of variance using Fisher's PLSD-test, and a P value of less than 0.05 was considered significant.

### Results

### Effect of Dapsone on NHBE Cell Viability.

To confirm that the total number of cultured NHBE cells was not influenced by dapsone treatment, the viability of cells was evaluated using CCK-8 (Dojindo). NHBE cells seeded onto 96-well plates (3,000 cells/well) were cultured at 37° C. for 72 h (˜70% confluence). Dapsone at concentrations of 0.3, 1 or 10 μg/ml was added for 24 or 72 h. The results showed that, for cells treated with dapsone, the total viable cell number was similar to that of the non-treated control group over 72 h (not shown).

### Effect of Dapsone on LPS-Induced IL-8 Secretion.

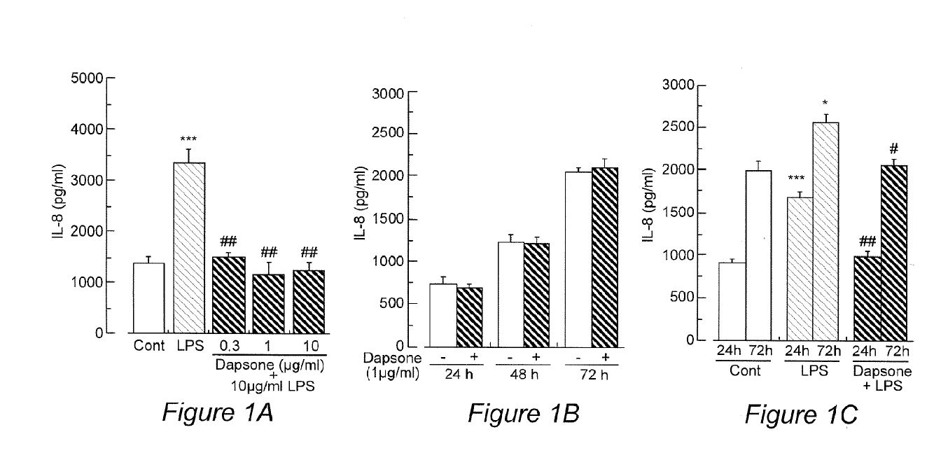

To evaluate the effect of dapsone on IL-8 secretion from NHBE cells, cell supernatants were harvested 24 h after stimulation with LPS in the presence and absence of dapsone, which had been added at the same time as the LPS. We chose to assess cell response at the time of cell confluence rather than normalize to the relative number of cells, because cell maturation could potentially affect cell signaling and cytokine secretion, and at confluence, all cells are at similar growth stages. LPS at 10 μg/ml significantly increased IL-8 secretion from NHBE cells (P<0.001), but dapsone at 0.3, 1 or 10 μg/ml suppressed this (P<0.01 for each) (FIG. 1A). Dapsone 1 μg/ml did not affect basal IL-8 secretion for 48 and 72 h (FIG. 1B). In the presence of dapsone 1 μg/ml, LPS-induced IL-8 secretion was significantly and persistently decreased for up to 72 h (FIG. 1C).

### Effect of Dapsone in ALI-Conditioned NHBE Cells.

To confirm if the IL-8-inhibiting effect of dapsone is seen in differentiated cells, we used NHBE cells cultured under ALI condition for 14 days. For cells at ALI, samples were collected from both apical and basolateral chambers of cells grown on filters. To collect samples from the apical chamber, 150 μl of Hanks' balanced salt solution (HBSS; Lonza Walkersville, Inc.) was added to apical side. Apical IL-8 concentrations were expressed as values of four-fold dilution to be equal to the basolateral medium volume of 600 μl.

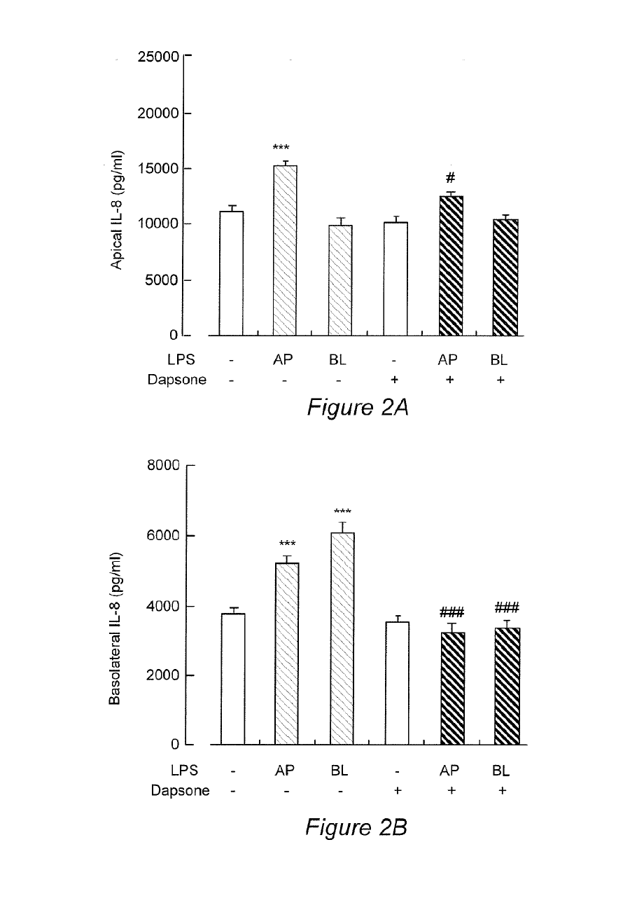

NHBE cells were stimulated with LPS from the apical (AP-LPS) or basolateral side (BL-LPS) for 24 h, and IL-8 secretion levels in both chambers were measured. Dapsone was added only to the basolateral medium. As shown in FIG. 2A, apical IL-8 level was significantly increased by AP-LPS (P<0.001), and dapsone inhibited this response (P<0.05). Likewise, basolateral IL-8 was significantly increased by AP- or BL-LPS (P<0.001 for each) (2B), and dapsone inhibited the both the apical and basolateral response (P<0.001 for AP-LPS, P<0.01 for BL-LPS).

To compare with corticosteroids, we examined the effect of dexamethasone (DEX) on LPS-induced IL-8 secretion. DEX suppressed basal IL-8 level in both apical and basolateral sides (P<0.05 for each) (FIG. 2C, 2D). DEX also significantly inhibited AP-LPS- or BL-LPS-induced increase in IL-8 (P<0.05 for each).

Effect of Dapsone on LPS-Induced IL-8 mRNA Expression.

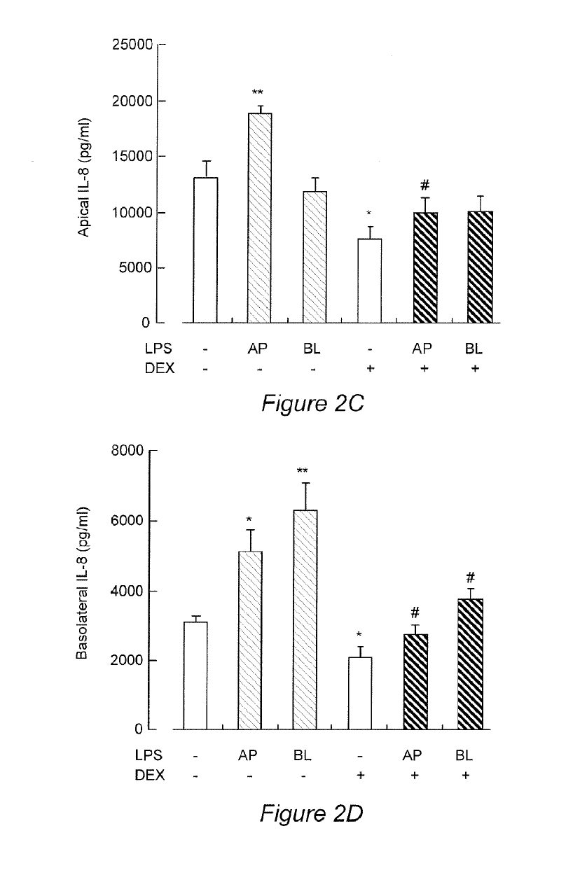

To examine the action of dapsone on IL-8 mRNA, RNA was extracted from the cells after 4 h stimulation of LPS, dapsone, DEX or their combination, and prepared for real-time quantitative PCR. As shown in FIG. 3, 1 μg/ml dapsone did not influence the basal IL-8 mRNA level, but 0.1 μg/ml DEX decreased this by −40% (P<0.05). 10 μg/ml LPS increased IL-8 mRNA level more than 5-fold of control (P<0.001). Dapsone at 1 and 10 μg/ml significantly inhibited LPS-induced IL-8 mRNA over expression (P<0.05 for each). DEX at 0.1 μg/ml also inhibited this (P<0.01).

### Effect of Dapsone on LPS-Induced Phosphorylation of MAPKs

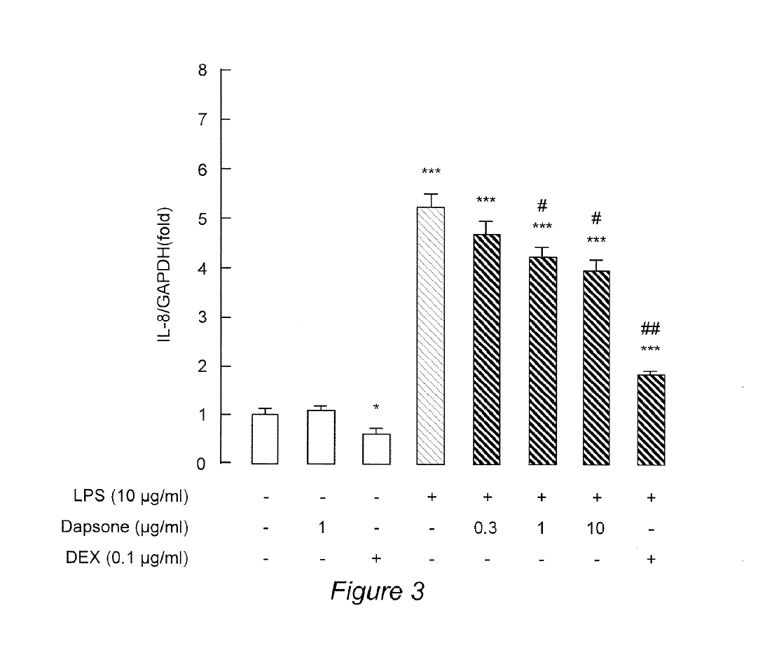

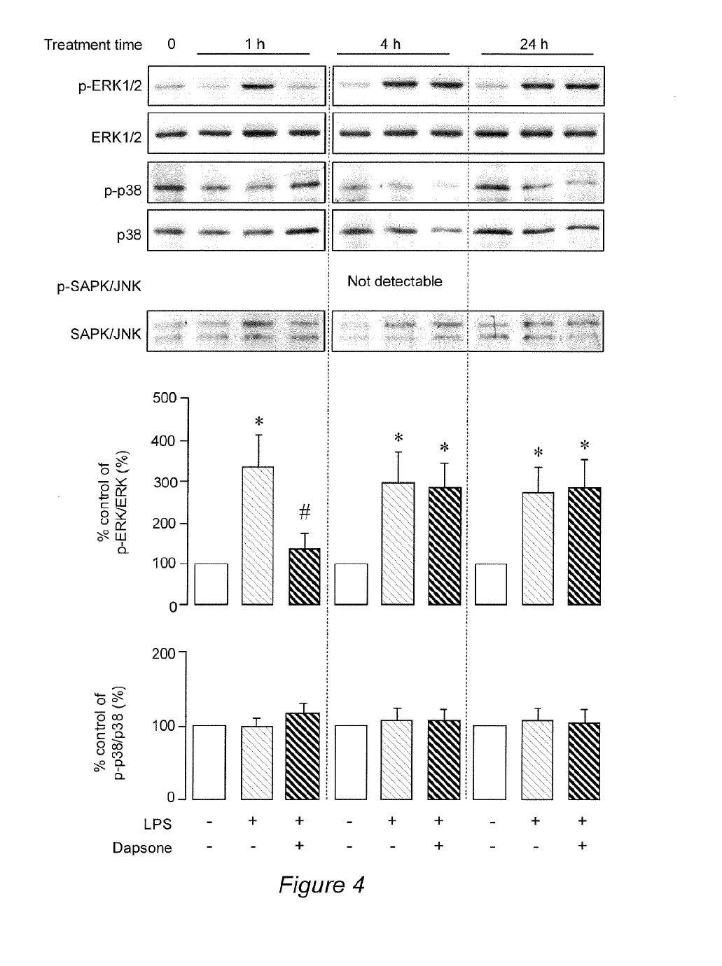

MAPK signaling are important pathways in the synthesis of IL-8. We evaluated the effect of dapsone on LPS-induced phosphorylation of ERK1/2, p38, and JNK in NHBE cells. LPS at 10 μg/ml significantly phosphorylated ERK1/2 at 1, 4, and 24 h (P<0.05 for each), but not p38 and JNK (FIG. 4). Dapsone at 1 μg/ml inhibited LPS-induced ERK1/2 phosphorylation at 1 h (P<0.05), although this effect disappeared after 4 h. We then assessed the effect of PD98059 (2′-amino-3′-methoxyflavone), a selective cell-permeable MEK inhibitor. As shown in FIG. 5A, LPS dose dependently increased ERK1/2 phosphorylation and IL-8 secretion, and PD98059 at 20 μM abolished 10 μg/ml LPS-induced ERK1/2 phosphorylation. However, this concentration of PD98059 did not inhibit IL-8 secretion (FIG. 5B).

### Effect of Dapsone on LPS-Induced Phosphorylation of NF-κB p65.

Since LPS simulates TLR4 and then induces IL-8 through the NF-κB pathway, we examined the effect of dapsone on NF-κB p65 phosphorylation in NHBE cells. Growth factors were withdrawn from culture medium 24 h before LPS or dapsone exposure. The threonine phosphorylation of NF-κB p65 was measured by Western blotting. The band intensity was calculated with NIH Image J software.

The results showed that LPS at 10 μg/ml significantly increased NF-κB p65 phosphorylation at 15 min (P<0.01), and this effect persisted up to 2 h (P<0.05). Dapsone at 1 μg/ml significantly inhibited LPS-induced NF-κB p65 phosphorylation to the control level (P<0.01 for 15 and 30 min, P<0.05 for 1 and 2 h). Further, this inhibitory effect was dose-dependent (P=0.16 for 0.3 μg/ml dapsone and P<0.01 for 1 and 10 μg/ml dapsone).

Effect of Tracheal LPS with and without Dapsone on Ferret Activity and Weight.

Ferrets stimulated with LPS for 5 days received 5-day dapsone treatment or vehicle alone (n=8 for vehicle, and n=9 for dapsone group each time point that was examined). There was no measurable effect of LPS with or without dapsone treatment on ferret activity or appetite and no difference in weight over 9 days when comparing these two groups (not shown).

### Intraepithelial Neutrophil Accumulation in LPS-Inflamed Ferret Trachea.

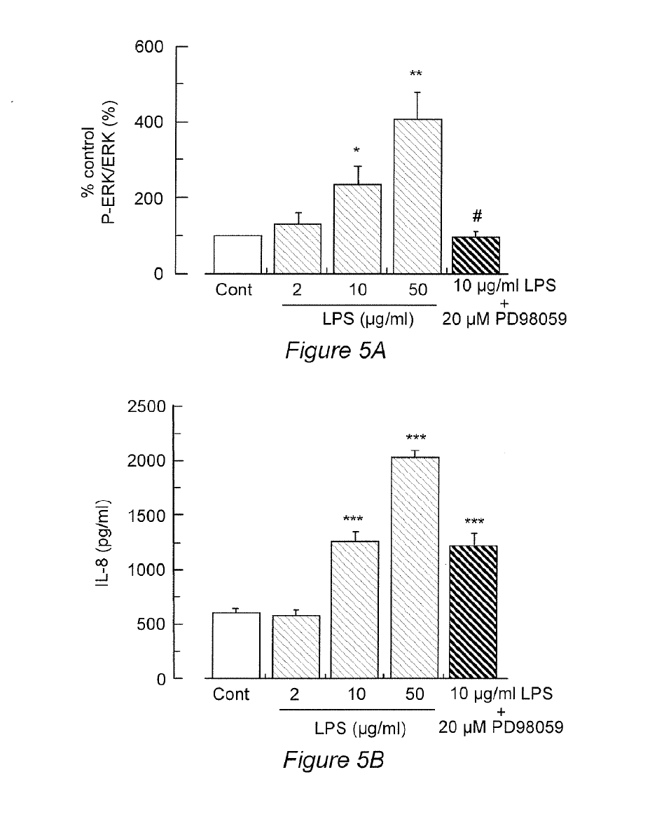

To evaluate in vivo effect of dapsone, we used topical LPS coated onto an endotracheal tube (ETT) to recruit neutrophils and inflame the trachea of anaesthetized and spontaneously breathing ferret (Abanses et al, 2009). Control ferrets intubated with an ETT coated with only a water soluble jelly (used as the LPS vehicle in the other group) showed little few epithelial neutrophils; less than 3/150 μm. One ferret with an LPS-inflamed airway and treated with nebulized vehicle was not completed because of death on day 6, and this animal was excluded from further analysis. LPS exposure induced marked neutrophil accumulation in the ferret tracheal epithelium and dapsone treatment reduced intraepithelial neutrophil number (not shown). Orally-administered dapsone tended to inhibit neutrophil recruitment (P=0.3) (FIG. 6A), and nebulized dapsone significantly inhibited neutrophil accumulation (P<0.05) (FIG. 6B).

### Mucociliary Transport (MCT) on Excised Tracheal Segments.

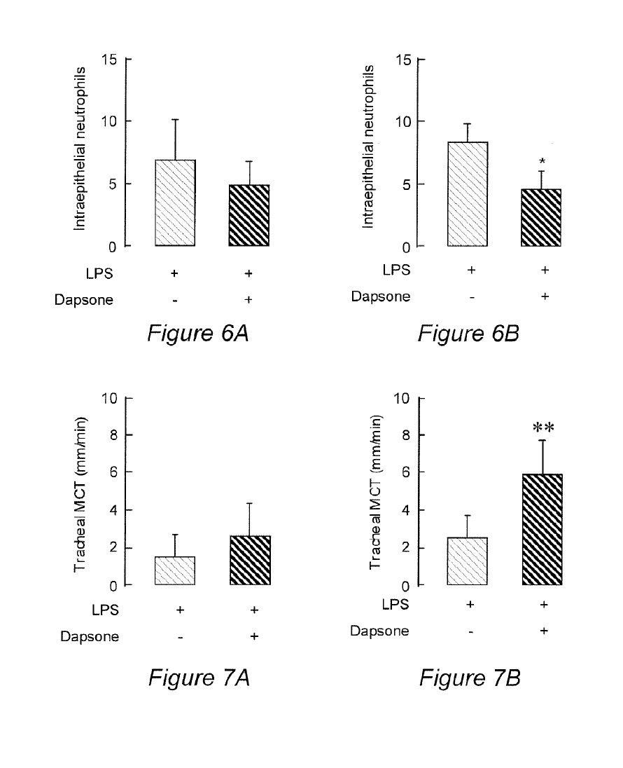

Mucociliary transport (or “mucociliary clearance”, MCC) is an overall measurement of the health and integrity of the airway surface. MCT was timed over a 3 mm segment. LPS dramatically decreased MCT to 1-3 mm/minute (normal is approximately 7 mm/min). Oral dapsone increased MCT but the increase was not significant (P=0.09). However aerosol dapsone preserved MCT at near normal velocity (6 mm/min; P=0.007 compared with LPS control) as shown in FIGS. 7A and B

### DISCUSSION

We have shown that dapsone inhibits IL-8 secretion from NHBE cells stimulated with LPS. Dapsone is used to treat dermatologic disorders, most notably those with neutrophil infiltrates. It has been postulated that dapsone impairs neutrophil chemotaxis and function at the sites of inflammation, apparently without increased risk of opportunistic infections. This is consistent with immunomodulation, but not immunosuppression.

Dapsone inhibits local production of toxic reactive oxygen species, myeloperoxidase and elastase, but this seems unlikely as a principal mode of action because the clinical response to dapsone is characterized by decreasing the neutrophil numbers. Other investigators have shown that dapsone may impair neutrophil chemotaxis by interfering with activation of adhesion molecule CD11b/CD18 in vitro. However, this seems to require a higher concentration of dapsone than therapeutic levels measured in vivo. The concentration of dapsone we used in these studies is within the ranges required for therapeutic serum levels of 0.5-5 μg/ml (Zhu et al, 2001). We found that the lower concentration of dapsone of 0.3 μg/ml inhibited LPS-induced IL-8 secretion and this effect was not due to cell toxicity as measured by the number of viable cells. Schmidt et al. (2001) reported that dapsone, in a therapeutic concentration, inhibited the bullous pemphigoid IgG-mediated IL-8 release from cultured normal human epidermal keratinocytes (NHEK) and that dapsone did not depress basal IL-8 level. These data are similar to our results using NHBE cells.

Airway epithelia are functionally polarized, and there is evidence that epithelial cells can secrete cytokines in a bidirectional manner. To better understand the mode of action of dapsone in differentiated and polarized airway epithelial model, NHBE cells were cultured at an ALI (Kanol et al., 2001). In the presence of dapsone, IL-8 secretion induced by LPS stimulation was significantly reduced, while constitutive IL-8 release was not inhibited by dapsone. Interestingly, when stimulated apically (as would occur during an airway infection) NHBE cells secreted IL-8 toward apical as well as basolateral sides, whereas when stimulated basolaterally they secreted only towards the basolateral side. Airway epithelial cells grown on filters and stimulated by Staphylococcus aureus at the apical side caused T cells chemotaxis towards the apical but not the basal side supernatant (Escotte et al, 2006). The presence of an apically oriented chemoattractant gradient may be necessary to drive immune cells like neutrophils and lymphocytes across epithelial surfaces (Chin et al, 2007). Additionally, we tested the effect of dexamethasone (DEX), a potent synthetic corticosteroid, in our cultured cell system. DEX is reported to inhibit IL-8 release from NHBE cells, and we confirmed that DEX inhibited IL-8 mRNA expression more than dapsone. However unlike dapsone, DEX suppressed basal IL-8 level as well as LPS-induced increase, suggesting that DEX is immunosuppressive while dapsone is immunomodulatory.

The intracellular signaling pathways by which dapsone inhibits IL-8 have not been well characterized. Because MAPK regulates IL-8 gene expression in airway epithelial cells, we evaluated the effect of dapsone on these pathways. We found that LPS dose dependently increased ERK1/2 phosphorylation, but not JNK and p38, in NHBE cells, and elevated p-ERK1/2 level continued for at least 24 h. We have previously shown that sustained activation of ERK1/2 is required for basal IL-8 secretion from unstimulated NHBE cells, and that a specific MEK inhibitor PD98059 suppressed IL-8, while a pharmacological inhibitor of JNK or p38 did not. Others have shown that pretreatment with PD98059 inhibits LPS-induced phosphorylation of ERK1/2 at 1 and 2 h. In the current study, the inhibitory effect of PD98059 was confirmed even at 4 h. However, PD98059 did not decrease LPS-induced IL-8 secretion. Dapsone at 1 μg/ml inhibited p-ERK1/2 at 1 h, but not after 4 h. Taken together, it is unlikely that ERK1/2 inhibition alone will suppress IL-8 in LPS-stimulated cells, and therefore that temporal ERK1/2-inhibition by dapsone does not account for the effect on IL-8.

We then examined the effect of dapsone on NF-κB activation. NF-κB can induce gene expression of inflammatory mediators and cytokines in airway epithelial cells, and LPS can activate NF-κB via TLR4. The basic NF-κB complex is a dimer of two members of the Rel family proteins, p50 and p65 (RelA). Both subunits contact DNA, but only p65 contains a transactivation domain within the C-terminal region that directly interacts with the transcription apparatus. NF-κB p65 is activated by phosphorylation, which enhances its transcriptional activity, and is associated with nuclear translocation. We showed that LPS at 10 μg/ml elicited significant NF-κB p65 phosphorylation from 15 min to 2 h after stimulation in NHBE cells. Similar kinetics in response to LPS has been shown in murine intestinal myofibroblasts, in which phosphorylation of NF-κB p65 was detected within 30 min of LPS treatment and slowly decreased over 4 h. We found that dapsone at 1 μg/ml inhibited LPS-induced NF-κB p65 phosphorylation over 2 h to basal levels, suggesting that dapsone may inhibit induction of IL-8 secretion by blocking NF-κB p65 phosphorylation. Also the dose-dependent inhibitory effect on phospho-NF-κB p65 was consistent with results of experiment in IL-8 mRNA expression. Accordingly, the action of dapsone is due, at least in part, by down-regulating IL-8 at gene transcription. However, while dapsone 0.3 μg/mL did not significantly inhibit NF-κB p65 activation, the same concentration of dapsone strongly inhibited IL-8 release. More than 1 g/ml of dapsone was needed to inhibit LPS-induced IL-8 mRNA expression. Schmidt et al. (28) speculated that dapsone inhibits IL-8 release from NHEK at the post-transcriptional level without affecting mRNA concentration.

These immunomodulatory effects were similar to the immunomodulatory effects of macrolides such as erythromycin, clarithromycin and azithromycin. Clarithromycin modulates ERK1/2 phosphorylation in NHBE cells, and azithromycin inhibits NF-icB activity in a CF cell line; thus regulating IL-8. It is likely that dapsone also modulates airway inflammation via modulation of IL-8.

After inducing tracheal inflammation in ferrets using topically applied LPS, we evaluated the effects of five days of oral or aerosol dapsone on neutrophil inflammation and MCT, an integrated measure of epithelial integrity. Oral dapsone decreased intraepithelial neutrophil accumulation but this was not statistically significant. Recognizing that dapsone is effective when used topically as a cream to treat neutrophilic dermatoses, we evaluated the potential for a lower dose of aerosol dapsone to inhibit LPS-induced tracheal inflammation. Nebulized dapsone significantly inhibited airway neutrophil infiltration and preserved or restored MCT despite exposure to LPS. Although it is difficult to calculate the precise dose to the airway, even if 10% of the total dose is delivered efficiently to the trachea (38), the dapsone delivered was only 0.3 mg. This dose is approximately one-tenth the systemic dose administered orally in a 1.5 kg ferret.

In summary, dapsone did not influence unstimulated (basal) IL-8 secretion. Apical LPS stimulation induced both apical and basolateral IL-8, but basolateral LPS increased only basolateral IL-8. Dapsone inhibited polarized IL-8 secretion from ALI-conditioned cells. Dapsone also decreased LPS-induced IL-8 mRNA levels. LPS led to phosphorylation of extracellular signal regulated kinase (ERK)1/2, but not p38 MAPK or c-Jun N-terminal kinase. LPS also induced NF-κB p65 phosphorylation, an effect that was inhibited by dapsone. Both oral and aerosol dapsone decreased LPS-induced intraepithelial neutrophil accumulation but only treatment with aerosol dapsone restored mucociliary transport to normal.

Together, these data show that dapsone inhibits IL-8 in human airway cells and neutrophil recruitment in the inflamed mammalian trachea in vivo while preserving MCT. Aerosol dapsone could be a promising therapy to treat chronic inflammatory airway diseases such as cystic fibrosis, chronic bronchitis, or severe asthma. Thus, dapsone, given either systemically or especially as an aerosol, may be useful in treating neutrophilic airway inflammation.

### Example 2. IL-13 Inhibiting Properties of Dapsone

Globlet cells are columnar epithelial cells whose sole function is to secrete mucin, which dissolves in water to form mucus. Goblet cell hyperplasia is involved in the pathological hypersecretion exhibited by bronchial epithelial cells of asthmatics, and IL-13 is known to pa central role in mediating goblet cell hyperplasia in both in vivo and in vitro models of asthma. The ability of dapsone to inhibit goblet cell hyperplasia was tested in vitro. An in vitro model of goblet cell hyperplasia was developed using normal human bronchial epithelial (NHBE) cells cultured under air-liquid interface (ALI) conditions. Control experiments that were analyzed using differential cell staining and microscopy showed that dapsone (3 μg/ml) had no measurable effect on the growth of ALI-conditioned NHBE cells (not shown). However, when goblet cell hyperplasia was induced using IL-13, dapsone (10 μg/ml) decreased the amount and extent of goblet cell hyperplasia, compared to control cell cultures (not shown). Thus, dapsone may exert its beneficial effect on resolving inflammation by also inhibiting IL-13 induced goblet cell hyperplasia.

### REFERENCES

Abanses J C, Arima S, Rubin B K. Vicks VapoRub induces mucin secretion, decreases ciliary beat frequency, and increases tracheal mucus transport in the ferret trachea. Chest 2009; 135:143-148.
Berlow, B A, Liebhaber, M I, Zeb, D and Spiegel, T M. J Allergy Clin Immunol. 1991 March; 87(3):710-715.
Chin A C, Parkos C A. Pathobiology of neutrophil transepithelial migration: implications in mediating epithelial injury. Annu Rev Pathol 2007; 2:111-143.
Chougule, M, Padhi, B and Misra, A. AAPS Pharm Sci Tech, 9(1): 47-53.
Schmidt E, Reimer S, Kruse N, Bröcker E B, Zillikens D. The IL-8 release from cultured human keratinocytes, mediated by antibodies to bullous pemphigoid autoantigen 180, is inhibited by dapsone. Clin Exp Immunol 2001; 124:157-162.
Escotte S, Al Alam D, Le Naour R, Puchelle E, Guenounou M, Gangloff S C. T cell chemotaxis and chemokine release after Staphylococcus aureus interaction with polarized airway epithelium. Am J Respir Cell Mol Biol 2006; 34:348-354.
Kanoh S, Kondo M, Tamaoki J, Kobayashi H, Motoyoshi K, Nagai A. Differential regulations between adenosine triphosphate (ATP)- and uridine triphosphate-induced Cl-secretion in bovine tracheal epithelium. Am J Respir Cell Mol Biol 2001; 25:370-376.
Zhu Y I, Stiller M J. Dapsone and sulfones in dermatology: overview and update. J Am Acad Dermatol 2001; 45:420-434.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for preventing or treating inflammation in an airway of a patient in need thereof, comprising the step of
administering to said airway of said patient a formulation comprising dapsone, wherein said formulation is an aerosolized formulation or an aqueous formulation.

2. The method of claim 1, wherein said formulation is an aerosolized formulation and said step of administering is carried out using a device selected from the group consisting of a metered-dose inhaler and a dry powder inhaler.

3. The method of claim 1, wherein said formulation is an aqueous formulation and said step of administering is carried out via installation.

4. The method of claim 1, wherein said patient is suffering from a disease or condition selected from the group consisting of cystic fibrosis, bronchiectasis, bronchiolitis obliterans, emphysema, chronic bronchitis, chronic rhinosinusitis, toxic inhalation injury, chronic obstructive pulmonary disease, idiopathic pulmonary fibrosis, asthma, and chronic airway inflammation.

5. A method of preventing or treating abnormal mucociliary transport in an airway of a patient in need thereof, comprising the step of
administering to said airway of said patient a formulation comprising dapsone, wherein said formulation is an aerosolized formulation or an aqueous formulation.

6. The method of claim 5, wherein said formulation is an aerosolized formulation and said step of administering is carried out using a device selected from the group consisting of a metered-dose inhaler and a dry powder inhaler.

7. The method of claim 5, wherein said formulation is an aqueous formulation and said step of administering is carried out via installation.

8. The method of claim 5, wherein said patient is suffering from a disease or condition selected from the group consisting of cystic fibrosis, bronchiectasis, bronchiolitis obliterans, emphysema, chronic bronchitis, chronic rhinosinusitis, toxic inhalation injury, chronic obstructive pulmonary disease, idiopathic pulmonary fibrosis, asthma, and chronic airway inflammation.
